(19) United States
(12) Patent Application Publication
(10) Pub. No.: US 2012/0149715 A1
(43) Pub. Date: Jun. 14, 2012

(54) COMPOUNDS AND METHODS FOR THE TREATMENT OF VIRAL INFECTIONS

(76) Inventors: Yi Tsun Richard Kao, Hong Kong (CN); Kwok-Yung Yuen, Hong Kong (CN); Dan Yang, Hong Kong (CN); Lihong Hu, Lund (SE); Guanhua Chen, Hong Kong (CN); Bo-Jian Zheng, Hong Kong (CN)

(21) Appl. No.: 13/149,059

(22) Filed: May 31, 2011

Related U.S. Application Data
(60) Provisional application No. 61/349,525, filed on May 28, 2010.
(60) Provisional application No. 61/349,565, filed on May 28, 2010.

(51) Int. Cl. A61K 31/496; G06G 7/48; A61P 31/16; C07D 413/06; A61P 31/12

(52) U.S. Cl. 514/254.04; 544/367; 703/11

(57) ABSTRACT

High throughput and virtual screening methods are disclosed that can identify potential anti-viral agents. The virtual screening methods identify agents that interact with a viral nucleoprotein binding site. The high throughput methods identify compounds that inhibit viral infection by binding to viral nucleoprotein. Also disclosed are pharmaceutical formulations useful for treating or preventing viral infections, especially influenza A.

### CROSS-REFERENCE TO RELATED APPLICATIONS

This application claims priority to U.S. Ser. No. 61/349,525 entitled “Compounds and Methods for the Treatment of Viral Infections”, filed May 28, 2010, and U.S. Ser. No. 61/349,565 entitled “Compounds and Methods for the Treatment of Proliferative Diseases”, filed May 28, 2010, the contents of both being incorporated herein by reference.

### FIELD OF THE INVENTION

The present invention relates to methods of identifying compounds for the treatment or prevention of viral infections, in particular compounds that bind to the nucleozin binding site of a viral influenza nucleoprotein, and methods of making and using thereof.

### REFERENCE TO SEQUENCE LISTING

The Sequence Listing submitted May 31, 2011 as a text file named “UHK_{—}00358_ST25.txt,” created on Mar. 31, 2011, and having a size of 27,164 bytes is hereby incorporated by reference pursuant to 37 C.F.R. §1.52(e)(5).

### BACKGROUND OF THE INVENTION

Influenza is caused by an RNA virus of the orthomyxoviridae family. There are three types of influenza viruses: A, B and C. Influenza A viruses infect mammals (e.g. humans, pigs, ferrets, horses) and birds. Influenza A viruses are a global health concern, and have been responsible for three major pandemics that have killed over 50 million people worldwide since 1900. For example, the devastating “Spanish flu” (H1N1 influenza A virus) in 1918 killed more than twenty million people worldwide. Subsequent pandemics, including the Asian flu pandemic in 1957 (H2N2), the Hong Kong flu pandemic in 1968 (H3N2), the re-emergence of H1N1 (Russian flu) in 1970, along with the avian flu virus H5N1 in 1997 and 2003, suggest that pandemic influenza or possible bioterrorist attacks with flu viruses remains a major threat to global health and safety. Despite the profound effects of influenza viruses on public health throughout history, the standard treatments for influenza infections still remain inadequate.

The most common targets for small molecule-based therapeutics to combat influenza virulence include the proton-selective M2 ion channel and the protein neuramidase (NA). The M2 ion channel is integral to the maintenance of the viral envelope of the influenza A virus, while NA promotes budding of nascent viral particles from the host cell. Resistance is common among inhibitors directed at both targets, and has become widespread in clinical isolates. Almost 100% of the 2008 influenza H1N1 virus (swine flu) samples were resistant to the neuramidase inhibitor oseltamivir (Tamiflu), while more than 90% of the H3N2 viruses were resistant to M2 channel blocker adamantanes.

Besides resistance, factors including mode of administration and environmental impact affect the development of effective influenza treatments. For instance, Zanamivir (Relenza) can only be administered by inhalation and may not reach infected lung tissue that is poorly aerated. The widely used and stockpiled drug Oseltamivir is not degraded during the course of normal sewage treatment.

There is a need for a method of identifying compounds which inhibit viral replication of influenza strains in vitro and in vivo. There is a further need for antiviral formulations that inhibit influenza replication and reduce virulence of the influenza infection and/or prevent influenza infection.

Therefore, it is an object of the present invention to provide assays for identifying compounds that effectively interact with nucleoproteins (NPs). It is a further object of the invention to provide methods of making and using small molecule inhibitors of influenza A nucleoprotein (NP).

It is a still further an object of the invention to provide pharmaceutical compositions that effectively treat or prevent influenza A viral infections.

### SUMMARY OF THE INVENTION

Methods have been developed to identify potential anti-proliferative agents using high throughput screening and virtual screening. The high throughput screening method is specific for compounds that bind to influenza A nucleoprotein (NP) in cell-based or cell-free systems. The virtual screening methods identify compounds that may bind to a nucleoprotein. Both methods identify anti-viral agents that interact with binding sites on the viral nucleoprotein. In preferred embodiments, the screening methods are specific for compounds that bind to the nucleozin site of the influenza A NP.

Also disclosed are compounds according to formula I below:

Ar¹—Y—Ar²—X-Cy-Z—Ar³ (Formula I)

wherein Ar¹, Ar², and Ar³ are each independently substituted or unsubstituted aryl or heteroaryl groups;

X, Y, and Z are independently absent (i.e, a direct bond) or selected from —C(═O)—, —S(═O)—, —SO₂—, —C(═O)N(R₁), —N(R₂)—, —C(R₃)═C(R₄)—, and —C(R₅R₆)ₙ—,

wherein n is 0 to 10, preferably 0 to 6, and

wherein R₁-R₆ are each independently selected from hydrogen, halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl; and

Cy is a 5-7 membered substituted or unsubstituted cyclic or heterocyclic group, and methods of making thereof.

Methods of treating and/or preventing viral infections by administering a compound that inhibits nuclear accumulation of NP or binds to a viral nucleoprotein are also described herein. In a preferred embodiment, compounds and/or formulations are used to treat influenza infection, in particular influenza A infections. Preferred influenza strains to be treated include H1N1, H3N2, and H5N1. The compounds can be administered enterally or parenterally. In a preferred embodiment, the compounds are formulated orally for administration to a patient in need thereof.

### BRIEF DESCRIPTION OF THE DRAWINGS

FIG. 1 shows a dose-response curve for nucleozin-treated mammalian cells infected with influenza A H1N1, H3N2, and H5N1 strains, graphing the percent plaque forming units (“PFU”) relative to controls in the absence of nucleozin as a function of the concentration of nucleozin (μM) for H1N1 (A/WSN/33) (filled circles), H3N2 (local clinical isolated) (open circles), and H5N1 (A/Vietnam/1194/04) (filled upside triangles).

FIG. 2 shows a survival curve for nucleozin-treated (filled square) or untreated mice (open triangle) when challenged with the highly pathogenic A/Vietnam/1194/04 H5N1 virus.

### DETAILED DESCRIPTION OF THE INVENTION

### I. Definitions

“Alkyl” as generally used herein refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl generally has 30 or fewer carbon atoms in its backbone (e.g., C1-C30 for straight chain, C3-C30 for branched chain), preferably 20 or fewer, preferably 10 or fewer, more preferably 6 or fewer, most preferably 5 or fewer. If the alkyl is unsaturated, the alkyl chain generally has from 2-30 carbons in the chain, preferably from 2-20 carbons in the chain, preferably from 2-10 carbons in the chain, more preferably from 2-6 carbons, most preferably from 2-5 carbons. Likewise, preferred cycloalkyls have from 3-20 carbon atoms in their ring structure, preferably from 3-10, preferably from 3-6, carbon atoms in their ring structure, most preferably 5, 6 or 7 carbons in the ring structure. Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadien yl, 3-(1,4-pentadienyl), ethynyl, 1- and 3-propynyl, and 3-butynyl.

The term “alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Aryl,” as generally used herein, refers to a carbon based aromatic ring having 3-20, preferably 5-15, more preferably 6-10 ring members, including phenyl, biphenyl, or naphthyl. The aryl group can be optionally substituted with one or more moieties selected from the group consisting of hydroxyl, acyl, amino, halo, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al. Protective Groups in Organic Synthesis, John Wiley and Sons, Third Edition, 2002. The term “aryl” includes one or more substitutions at one or more carbon atoms of the hydrocarbon radical. Suitable substituents include, but are not limited to, halogens, such as fluorine, chlorine, bromine, or iodine; hydroxyl; —R₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Binding pocket” or “binding site” as generally used herein refer to a region of a molecule or molecular complex that, as a result of its configuration, favorably associates with, or is occupied by, a moiety or region of the same molecule or molecular complex, or a moiety or region of a different molecule, molecular complex, and/or chemical compound. As will be appreciated by those of skill in the art, the nature of the cavity within a binding pocket will vary from molecule to molecule.

“Effective amount” as generally used herein refers to an amount, or dose, within the range normally given or prescribed to demonstrate an anti-viral effect, e.g., in vitro or in vivo. The range of an effective amount may vary from individual to individual; however, the optimal dose is readily determinable by those of skill in the art depending upon the use. Such ranges are well established in routine clinical practice and will thus be readily determinable to those of skill in the art. Doses may be measured by total amount given (e.g. per dose or per day) or by concentration. Doses of 0.01, 0.05, 0.1, 0.5, 1, 2, 3, 4, 5, 6, 7, 8, 9, 10, 15, 20, 25, 30, 40, 50, 100, 500 and 1000 mg/kg/day may be appropriate for treatment.

“Nucleozin binding site” as generally used herein refers to a site on influenza nucleoprotein (NP) A located in the body domain on the back of influenza A NP. In this conformation the nucleozin is located between residues 280 to 311 in the groove. Those skilled in the art will appreciate that the nucleozin binding site is slightly different depending on the compound bound therein and can incorporate other contacts in place of and/or in addition to the ones disclosed herein.

“Heterocycle” or “heterocyclic” as generally used herein refers to one or more rings of 5-12 atoms, preferably 5-7 atoms, with or without unsaturation or aromatic character and having at least one ring atom which is not a carbon. Preferred heteroatoms include sulfur, oxygen, and nitrogen. Multiple rings may be fused, as in quinoline or benzofuran. Particularly preferred heterocycle groups are 5-10-membered rings with 1-3 heteroatoms selected from O, S, P, Si, As, and N. Heterocycles include, but are not limited to azolidine, pyrrole, oxolane, furan, thiolane, thiophene, phospholane, phosphole, silane, silole, arsolane, arsole, imidazoline, pyrazolidine, imidazole, imidazoline, pyrazole, pyrazoline, oxazolidine, isoxazolidine, oxazole, oxazoline, isoxazole, isoxazoline, thiazolidine, isothiazolidine, thiazole, thiazoline, isothiazole, isothiazoline, dioxolane, oxathiolane, dithiolane, thiazole, dithiazole, furazan, oxadiazole, thiadiazole, tetrazole, piperidine, pyridine, pyran, tetrahydropyran, thiane, thiopyran, piperazine, diazine, morpholine, oxazine, thiazine, dithiane, dioxane, dioxin, triazine, trioxane, tetrazine, azapane, azepine, oxepane, oxepine, thiepane, thiepine, azocane, azocine, oxecane, and thiocane. Heterocycle or heterocyclic also refers to substituted rings, as defined in “aryl” or “alkyl.”

The term “heterocycle” includes one or more substitutions at one or more carbon or heteroatoms. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Heteroaryl” as generally used herein refers to an aromatic group having 3-20, preferably 5-15, more preferably 6-10 ring members and containing from one to four N, O, P, Si, As, or S atoms(s) or a combination thereof, which heteroaryl group is optionally substituted at carbon or nitrogen atom(s). Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5 membered heteroaryls having two heteroatoms in 1, 2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. Particularly preferred heteroaryl groups are 5-10-membered rings with 1-3 heteroatoms selected from O, S, and N.

The term “heteroaryl” includes one or more substitutions at one or more carbon or heteroatoms atoms. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Hits” as generally used herein refers to a compound which shows the desired activity or potency in a screening assay. For example, a hit compound forms a low energy, stable complex when bound to a NP binding site in silico.

“Influenza A” as generally used herein refers to mammalian Influenza A virus, e.g., H3N2, H1N1, H2N2, H7N7 and H5N1 (avian influenza virus) strains and variants thereof.

“Low energy, stable complex” as generally used herein refers to a complex in which a drug is bound in the binding site of the nucleoprotein by weak to strong intermolecular forces including, but not limited to, covalent bonds, hydrogen bonds, disulfide bonds, salt bridges, ionic bonds, metal coordination, hydrophobic forces, van der Waals interactions, cation-pi interactions, pi-stacking, and combinations thereof.

“Nucleoprotein” or “NP” as generally used herein refers to any protein that is structurally associated with nucleic acid. Exemplary nucleoproteins are identified and sequenced in certain strains of influenza viruses. The sequences of many nucleoproteins can be found in the NCBI database. The GenBank accession numbers of some exemplary NP sequences from influenza type A for subtype H1N1 are NP 040982 (AAA43467) (SEQ ID NO: 5 AND SEQ ID NO: 6), for subtype H3N2 are AAZ38620 (YP308843) (SEQ ID NO: 7 AND SEQ ID NO: 8); and for subtype H5N1 are AY856864 (SEQ ID NO: 9 AND SEQ ID NO: 10) and AAF02400 (SEQ ID NO: 11 AND SEQ ID NO: 12).

“Nucleozin” as generally referred to herein has the chemical structure as follows:

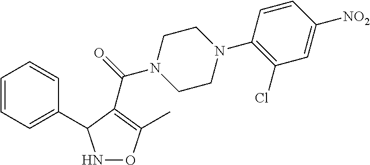

“Pharmaceutically acceptable” as generally used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable salts” as generally used herein refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic.

“Substituted” as generally used herein refers to a moiety (e.g., an alkyl group) substituted with one or more substituents including, but not limited to: halogen (e.g., fluorine, chlorine, bromine, and iodine); hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, tertiary, or quaternary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; alkyl, alkenyl, alkynyl, alkoxy, cycloalkyl, cycloalkenyl, heterocyloalkyl, or heterocycloalkenyl, aryl or heteroaryl.

“Substituted aryl” as generally used herein refers to aryl groups having one or more non-interfering groups as a substituent. For substitutions on a phenyl ring, the substituents may be in any orientation (i.e., ortho, meta, or para).

“Test compound(s)” as generally used herein refers to new or known small molecules (or libraries of molecules) subjected to the one or more assays described herein.

### II. Methods of Identifying Anti-Viral Agents that Interact with Viral Nucleoprotein by Virtual Screening

In a preferred embodiment, compounds that bind to form a stable, low energy complex with a nucleoprotein (NP) are identified by an in silico screen.

A virtual screening method that identifies potential anti-viral compounds that bind to a nucleoprotein binding site includes:

a.) obtaining the structural coordinates of a nucleoprotein;

b.) applying a 3-dimensional molecular modeling algorithm to the structural coordinates of the nucleoprotein binding pocket; and

c.) electronically screening stored spatial coordinates of the compound against the spatial coordinates of the nucleoprotein binding pocket to determine if the compound binds within the nucleoprotein binding pocket,

wherein a compound identified by the electronic screening as a compound that binds the viral nucleoprotein is identified as a compound that may bind to the viral nucleoprotein.

In preferred embodiments, virtual screening can be used to identify potential anti-viral agents that bind to the binding sites of influenza A NP.

A virtual screening method of identifying potential anti-viral compounds that may bind to influenza A NP nucleozin binding site includes:

a.) obtaining the structural coordinates of a influenza A NP;

b.) applying a 3-dimensional molecular modeling algorithm to the structural coordinates of an influenza A NP binding pocket defined by the structural coordinates of at least amino acid residues 280-311; and

(c) electronically screening stored spatial coordinates of the compound against the spatial coordinates of the influenza A NP binding pocket to determine if the compound binds within the influenza A NP binding pocket,

wherein a compound identified by the electronic screening as a compound that binds influenza A NP is identified as a compound that may bind to influenza A NP.

In some embodiments, libraries of small molecules can be docked into known or unknown binding sites of a viral nucleoprotein. In a preferred embodiment, the small molecules are docked into the nucleozin binding site of the influenza A NP.

The three dimensional structures of viral nucleoproteins are conserved between related strains. Accordingly, compounds which are identified to potentially bind to one particular nucleoprotein either in vitro, in vivo, or in silico can be screened in silico against other viral nucleoproteins to assess compound selectivity.

Protein structures for a number of nucleoproteins can be found in the Protein Data Bank, including structures for influenza A NP. Although some residues of the viral nucleoprotein may not be solved, homology modeling can be used to construct models of the NP. For example 2IHQ and 2Q06 can be used for homology modeling of H1N1 and H5N1 NP respectively using Swiss-Model homology.

In one embodiment, a computer model of a polypeptide consisting of a viral nucleoprotein binding pocket as defined herein is constructed using well-known software such as QUANTA [Molecular Simulations Inc, San Diego, Calif.], Sybyl [Tripos Associates, St. Louis, Mo.], InsightII [Accelrys], MOE [Chemical Computing Group Inc., Montreal, Quebec, Canada]. The preferred docking grid box for the influenza A NP has the coordinates X: 33.75 Å Y: 15.0 Å Z: 15.0 Å and is centered in the nucleozin-binding groove and covers the entire nucleozin binding site.

Selected compounds to be evaluated may then be positioned in a variety of orientations, or docked, within the binding pocket. Docking may be accomplished using software such as GRID, DOCK, AUTODOCK, FlexX, and GOLD. When a compound is docked within the binding pocket to form a “virtual” representation of drug-viral nucleoprotein complex, computational means may be further employed to generate quantitative and qualitative maps of the complex, including for example, pharmacophore maps, surface property maps (which map Conolly, Gaussian and van der Waals surfaces) and maps of Probabilistic Receptor Potentials using software such as QUANTA, Sybyl, InsightII, and MOE.

The efficiency with which a selected compound binds to the nucleoprotein binding pocket may be tested and optimized by computational evaluation. The quality of the fit of a given compound within binding pocket may be evaluated, for example, by shape, size and electrostatic complementarity as determined qualitatively by visual inspection or as determined quantitatively by the use of scoring functions such as LUDI, PLP, PMF, SCORE, GOLD and FlexX. These methods of qualitative and quantitative evaluation may be employed individually or in combination, for example, as in a consensus scoring manner.

Alternatively, binding efficiency can be determined based on the interaction energy of a complex formed by the binding or association of a compound with nucleoprotein. For example, a compound determined to form a “low energy, stable complex” with a viral nucleoprotein, in the manner described herein, warrants further analysis as a nucleoprotein inhibitor and anti-viral agent.

Potential intermolecular interactions which contribute to binding efficiency and formation of a low energy, stable complex include, but are not limited to, covalent bonds, hydrogen bonds, disulfide bonds, salt bridges, ionic bonds, metal coordination, hydrophobic forces, van der Waals interactions, cation-pi interactions, and pi-stacking.

Van der Waals interaction energy value can be determined using the software MOE, and is based on the MMFF94 force field. Accordingly, a compound determined to form a complex having a van der Waals interaction energy value of less than about 8000 kcal/mol is a potential anti-viral agent. Preferably, a low energy, stable complex in accordance with the present invention will have a van der Waals interaction energy value of less than about 6000 kcal/mol, and more preferably, a value of less than about 4000 kcal/mol.

In a preferred embodiment, the binding efficiency between the influenza A nucleoprotein and the compound is calculated. Compounds that form low energy, stable complexes with the influenza A nucleoprotein warrant further analysis as an influenza nucleoprotein A inhibitor and anti-viral agent. Preferred van der Waals interaction energies are less than about 800 kcal/mol, more preferably lower than 6000 kcal/mol, most preferably below 4000 kcal/mol.

Compound identified as hits by the virtual screen can be further evaluated using in vitro screens known in the art. For example, radiolabeled assays can be used to confirm that a particular compound is bound to the binding site.

### III. NP Inhibitory Formulations

### A. NP Inhibitory Compounds

In some embodiments, the compounds have the formulae I-V1 below, or pharmaceutically acceptable salts thereof.

In preferred embodiments, the NP inhibitors have the structure of formula I:

Ar¹—Y—Ar²—X-Cy-Z—Ar³ (Formula I)

wherein, Ar¹, Ar², and Ar³ are each independently substituted or unsubstituted aryl or heteroaryl groups;

X, Y, and Z are independently absent (i.e, a direct bond) or selected from —C(═O)—, —S(═O)—, —SO₂—, —C(═O)N(R₁), —N(R₂)—, —C(R₃)═C(R₄)—, and —C(R₅R₆)ₙ—;

n is 0 to 10, preferably 0-6; and

R₁-R₆ are each independently selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl; and

Cy is a 5-7 membered substituted or unsubstituted cyclic or heterocyclic group.

In some embodiments, Ar¹ is substituted with hydrogen, hydroxyl, nitro, amino, or azide; Ar² is substituted with a methyl group; X is C═O; Y and Z are absent; Cy is piperazine; and Ar³ is substituted with a halo group, a nitro group, or a combination of a halo and nitro group.

In some embodiments, Cy is a substituted 5-7 membered unsaturated ring containing 2 nitrogen atoms, wherein one nitrogen atom is bonded to X and another nitrogen atom is bonded to Z.

In a preferred embodiment, Cy is a substituted piperazine, wherein one nitrogen is bonded to X and the second nitrogen is bonded to Z.

In some embodiments, the NP inhibitors have the structure of formula II:

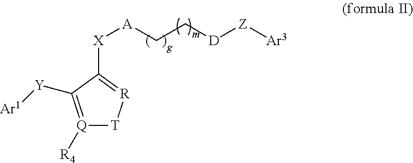

wherein Ar¹ and Ar³ are each independently substituted or unsubstituted aryl or heteroaryl groups;

X, Y, and Z are independently absent or selected from the group consisting of —C(═O)—, —S(═O)—, —SO₂—, —C(═O)N(R₁₀), —N(R₁₁)—, —C(R₁₂)═C(R₁₃)—, and —C(R₁₄R₁₅)ₙ—,

n, g, and m are independently 0 to 10, preferably 0-6;

T, Q, and R are, as valence and stability permit, independently selected from C(R₈R₉), nitrogen, oxygen, phosphorous, sulfur, selenium, boron, and arsenic;

A and D are each independently CR₁₆R₁₇ or NR₁₈;

wherein R₄ and R₈-R₁₈ independently are absent, or are selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl; or

—CR₁₆R₁₇—, —NR₁₈—, or combinations thereof, when taken together with the optional bridging methylene groups, form a 5-8-membered cyclic structure.

In some embodiments, Ar¹ substituted with hydrogen, hydroxyl, nitro, amino, or azide; X is —C═O; Y and Z are absent, and Ar³ is substituted with a halo group, a nitro group, or a combination of a halo and nitro group. In some embodiment, Ar¹ and Ar³ are phenyl rings and substituted as described above.

In a preferred embodiment, R₄ is methyl.

In some embodiments, Q is carbon, T is oxygen, and R is nitrogen.

In some embodiments, g and m are 1 and A and D are NR₁₇, wherein A-D defines a piperazine.

In some embodiments, the NP inhibitors have the structure of formula III:

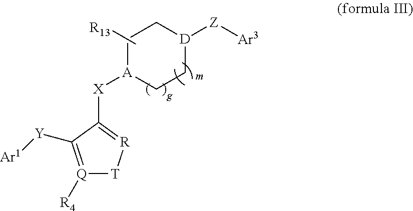

wherein Ar¹ and Ar³ are each independently substituted or unsubstituted aryl or heteroaryl groups;

X, Y, and Z are independently absent or selected from the group consisting of —C(═O)—, —S(═O)—, —SO₂—, —C(═O)N(R₁₀), —N(R₁₁)—, —C(R₁₂)═C(R₁₃)—, and —C(R₁₄R₁₅)ₙ—, n, g, and m are independently 0 to 10, preferably 0-6;

A, D, T, Q, and R are, as valence and stability permit, independently selected from C(R₈R₉), nitrogen, oxygen, phosphorous, silicon, sulfur, selenium, boron and arsenic;

wherein R₄ and R₈-R₁₅ independently are absent, or are selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl. One or more of R₁₃ can be present on the ring.

In some embodiments, Ar¹ is substituted with hydrogen, hydroxyl, nitro, amino, or azide; X is C═O; Y and Z are absent, and Ar³ is substituted with a halo group, a nitro group, or a combination of a halo and nitro group. In some embodiments, Ar¹ and Ar³ are phenyl rings substituted as described above.

In a preferred embodiment, Q is carbon, T is oxygen, and R is nitrogen.

In some embodiments, A and D are nitrogen.

In some embodiments, R₄ and R₁₃ are independently hydrogen or methyl.

In preferred embodiments, R₄ is methyl and R_{D} is hydrogen.

In some embodiments, the composition the NP inhibitors have the structure of formula IV:

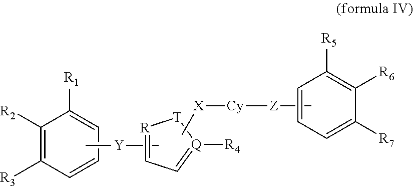

wherein X, Y, and Z are independently absent or selected from the group consisting of —C(═O)—, —S(═O)—, —SO₂—, —C(═O)N(R₁₀), —N(R₁₁)—, —C(R₁₂)═C(R₁₃)—, and —C(R₁₄R₁₅)ₙ—;

wherein n is 0 to 10, preferably 0-6;

T, Q, and R are, as valence and stability permit, independently selected from C(R₈R₉), nitrogen, oxygen, phosphorous, silicon, sulfur, selenium, boron, and arsenic; and

Cy is a 4-7 membered substituted or unsubstituted cyclic or heterocyclic group;

wherein R₁-R₁₅ independently are absent, or are selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl.

In some embodiments, Cy is a substituted 5-7 membered unsaturated ring containing 2 nitrogen atoms, wherein one nitrogen atom is bonded to X and another nitrogen atom is bonded to Z.

In a preferred embodiment, Cy is a substituted piperazine, wherein one nitrogen is bonded to X and the second nitrogen is bonded to Z, Y and Z are absent, X is C═O, T is oxygen, Q is carbon, and R is nitrogen.

In some embodiments, R₁-R₃ and R₅-R₇ are selected from a halo group, a nitro group, or a combination of a halo and nitro group.

In preferred embodiments, R₄ is a methyl group.

In some embodiments, the NP inhibitors have the structure of formula V:

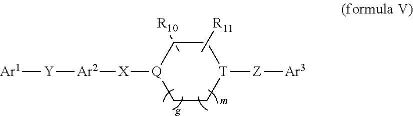

wherein Ar¹, Ar², and Ar³ are each independently substituted or unsubstituted aryl or heteroaryl groups

X, Y, and Z are independently absent or selected from the group consisting of —C(═O)—, —S(═O)—, —SO₂—, —C(═O)N(R₁), —N(R₂)—, —C(R₃)═C(R₄)—, and —C(R₅R₆)ₙ—;

n, g, and m are independently 0-10, preferably 0-6;

Q and T are independently selected from nitrogen or CR₇; and

R₁-R₇, R₁₀, and R₁₁ are independently selected from hydrogen; halogen; hydroxy; nitro; nitrile; isonitrile; urea; guanidine; cyano; carbonyl, such as formyl, acyl, or carboxyl; thiocarbonyl, such as thioester, thioacetate, or thioformate; primary, secondary, or tertiary amine (i.e., amino); amide; amidine; imine; azide; thiol, substituted or unsubstituted thioalkyl (e.g., thioether); isocyanate; isothiocyanate; phosphoryl; phosphate; phosphinate; sulfate; sulfonate; sulfamoyl; sulfonamide; sulfonyl; substituted or unsubstituted linear or branched alkyl, substituted or unsubstituted linear or branched alkenyl, substituted or unsubstituted linear or branched alkynyl, substituted or unsubstituted linear and branched alkoxy, substituted or unsubstituted C₃-C₁₀ cycloalkyl, cycloalkenyl, heterocyloalkyl, or heterocycloalkenyl, substituted or unsubstituted aryl or heteroaryl.

In some embodiments, Q and T are both nitrogen.

In some embodiments, R₁₀ is a methyl group and R₁₁ is hydrogen. In another embodiment, R₁₀ and R₁₁ are both hydrogen.

In some embodiments, Y and Z are absent and X is C═O.

In some embodiments, g and m are 1.

In a preferred embodiment, Ar¹ and Ar³ are a substituted phenyl, Ar² is a substituted isoxazole, Y and Z are absent, X is C═O, Q and T are nitrogen, g and m are 1, R₁₀ is methyl and R₁₁ is hydrogen.

In some embodiments, the NP inhibitors have the structure of formula VI:

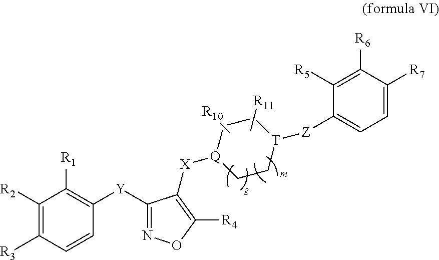

wherein X, Y, and Z are independently absent or selected from the group consisting of —C(═O)—, —S(═O)—, —SO₂—, —C(═O)N(R₁₂), —N(R₁₃)—, —C(R₁₄)═C(R₁₅)—, and —C(R₁₆R₁₇)ₙ—, n, g, and m are independently 0-10, preferably 0-6;

Q and T are independently selected from nitrogen or CR₁₈; and

R₁-R₁₈ are independently selected from hydrogen, halo, hydroxyl, linear or branched C₁-C₁₀, preferably C₁-C₆ alkyl, linear or branched C₁-C₁₁, preferably C₁-C₆ alkenyl, linear or branched C₁-C₁₀, preferably C₁-C₆ alkynyl, or linear and branched C₁-C₁₀, preferably C₁-C₆ alkoxy, amino, azide, cyano, nitro, nitrile, isonitrile, amide, carboxylate, urea, guanidine, isocyanate, isothiocyanate, and thioether.

In some embodiments, Q and T are both nitrogen.

In some embodiments, R₁₀ is a methyl group and R₁₁ is hydrogen. In other embodiments, both R₁₀ and R₁₁ are hydrogen.

In some embodiments, Y and Z are absent and X is C═O.

In some embodiments, g and m are 1.

In some embodiments, R₁-R₃ and R₅-R₇ are selected from a halo group, a nitro group, or a combination of a halo and nitro group.

In preferred embodiments, R₄ is a methyl group.

Some preferred compounds according to the invention are:
[4-(2-chloro-4-nitro-phenyl)-piperazin-1-yl]-[3-(4-hydroxy-phenyl)-5-methylisoxazol-4-yl]-methanone;
[4-(2-chloro-4-nitro-phenyl)-piperazin-1-yl]-[3-phenyl-5-methyl-isoxazol-4-yl]-methanone;
[4-(2-chloro-4-nitro-phenyl)-piperazin-1-yl]-[3-(4-amino-phenyl)-methylisoxazol-4-yl]-methanone;
[4-(2-chloro-4-nitro-phenyl)-piperazin-1-yl]-[3-(4-azido-phenyl)-5-methylisoxazol-4-yl]-methanone;
[4-(2-chloro-4-nitro-phenyl)-piperazin-1-yl]-[3-(2-chloro-phenyl)-5-methylisoxazol-4-yl]-methanone;
[4-(2-chloro-4-nitro-phenyl)-2-methyl-piperain-1-yl]-[3-(2-chloro-phenyl)-5-methyl-isoxazol-4-yl]-methanone;
[4-(2-chloro-4-nitro-phenyl)-2-methyl-piperain-1-yl]-[3-phenyl-5-methylisoxazol-4-yl]-methanone;
[4-(4-nitro-phenyl)-piperazin-1-yl]-[3-(2-chloro-phenyl)-5-methyl-isoxazol-4-yl]-methanone;
and [4-(4-nitro-phenyl)-piperazin-1-yl]-[3-(2,6-dichloro-phenyl)-5-methyl-isoxazol-4-yl]-methanone.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are known in the art.

The compounds may be co-administered with one or more additional active agents. Suitable compounds includes, but are not limited to, 13-cis-Retinoic Acid, 2-Amino-6-2-CdA, 2-Chlorodeoxyadenosine, Mercaptopurine, 5-fluorouracil, 5-FU, 6-TG, 6-Thioguanine, 6-Mercaptopurine, 6-MP, Accutane Actinomycin-D, Adriamycin, Adrucil, Agrylin, Ala-Cort, Aldesleukin, Alemtuzumab, Alitretinoin, Alkaban-AQ, Alkeran, All-transretinoi,c Alpha interferon, Altretamine acid, Amethopterin, Amifostine, Aminoglutethimide, Anagrelide, Anandron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium, Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine, Cytosar-U, Cytoxan, liposomal Dacarbazine, Dactinomycin, Darbepoetin, Daunomycin, Daunorubicin, Daunorubicin, Daunorubicin, DaunoXome hydrochloride, liposomal Decadron, Delta-Cortef, Deltasone, Denileukin, diftitox, DepoCyt, Dexamethasone, Dexamethasone, dexamethasone sodium acetate phosphate, Dexasone, Dexrazoxane, DHAD, DIC, Diodex, Docetaxel, Doxil, Doxorubicin, Droxia, DTIC, DTIC-Dome, liposomal Duralone, Efudex, Eligard, Ellence, Eloxatin, Elspar, Emcyt, Epirubicin, Epoetin, Erbitux, Erwinia, Estramustine, L-asparaginase, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluoxymesterone, Flutamide, Folinic Acid, FUDR, Fulvestrant, G-CSF, Gefitinib, Gemcitabine, Gemtuzumab, Gemzar, Gleevec, Gliadel wafer, Glivec, GM-CSF, Goserelin, Halotestin, Herceptin, Hexadrol, Hexylen, Hexamethylmelamine, HMM, Hycamtin, Hydrea, Hydrocort Acetate, Hydrocortisone, Hydrocortisone, Hydroxyurea, Ibritumomab, Ibritumomab, Idamycin, Idarubicin, Tiuxetan, Ifex, IFN-alpha, Ifosfamide, IL-2, IL-11, Imatinib mesylate, Imidazole, Interferon alpha, Carboxamide, Interferon alpha-2b, Interleukin-2, Interleukin-11, Iressa, Irinotecan, Isotretinoin, Kidrolase, Lanacort, L-asparaginase, LCR, Letrozole, Leucovorin, Leukeran, Leukine, Leuprolide, Leurocristine, Leustatin, Lomustine, L-PAM, L-Sarcolysin, Lupron, Lupron Depot, Matulane, Maxidex, Mechlorethamine, Mechlorethamine, Medralone, Medrol, Megestrol, Melphalan, Mercaptopurine, Mesna, Mesnex, Methotrexate, Methylprednisolone, Meticorten, Mitomycin, Mitomycin-C, Mitoxantrone M-Prednisol, MTC, MTX, Mustargen, Mustine, Mutamycin, Myleran, Mylocel, Mylotarg, Navelbine, Neosar, Neulasta, Neumega, Neupogen, Nilandron, Nilutamide, Nitrogen Mustard, Novaldex, Novantrone, Octreotide, Oncospar, Oncovin, Ontak, Onxal, Oprevelkin, Orapred, Orasone, Oxaliplatin, Paclitaxel, Pamidronate, Panretin, Paraplatin, Pegaspargase, Pegfilgrastim, PEG-INTRON, PEG-L-asparaginase, Phenylalanine, Platinol, Platinol-AQ, Prednisolone, Prednisone, Prelone, Procarbazine, PROCRIT, Proleukin, Prolifeprospan 20, Purinethol, Raloxifene, Rheumatrex, Rituxan, Rituximab, Roveron-A, Rubex, Rubidomycin, Sandostatin, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vincasar, Vincristine, Vinorelbine, VLB, VM-26, VP-16, Vumon, Xeloda, Zanosar, Zevalin, Zinecard, Zoladex, Zoledronic acid, Zometa, and pharmaceutically acceptable salts thereof.

### B. Formulations

Compounds that potentially bind to the nucleoprotein receptor binding site, and their pharmaceutically acceptable salts, can be formulated using standard techniques for enteral and parenteral administration. Preferred compounds are those that belong to formulae I-VI. Effective dosages can be determined based on the in vitro assays known to those skilled in the art, such as the assays described in the examples. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

1. Parenteral Formulations

The compounds described herein can be formulated for parenteral administration. “Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

i. Controlled Release Formulations

The parenteral formulations described herein can be formulated for controlled release including immediate release, delayed release, extended release, pulsatile release, and combinations thereof.

a) Nano- and Microparticles

For parenteral administration, the one or more NP inhibitors, and optional one or more additional active agents, can be incorporated into microparticles, nanoparticles, or combinations thereof that provide controlled release. In embodiments wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. Detailed descriptions of these processes can be found in “Remington—The science and practice of pharmacy”, 20th Edition, Jennaro et. al., (Phila, Lippencott, Williams, and Wilkens, 2000).

For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin (Cortesi, R., et al., Biomaterials 19 (1998) 1641-1649). Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water soluble protein can be spray coated onto the microparticles and subsequently cross-linked by the one of the methods described above. Alternatively, drug containing microparticles can be microencapsulated within protein by coacervation-phase separation (for example, by the addition of salts) and subsequently cross-linked. Some suitable proteins for this purpose include gelatin, albumin, casein, and gluten. Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

2. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

i. Controlled Release Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

Extended Release Formulations

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers. In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids. The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Formulations

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

3. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The compounds can also be formulated for intranasal delivery, pulmonary delivery, or inhalation. The compositions may further contain one or more chemical penetration enhancers, membrane permeability agents, membrane transport agents, emollients, surfactants, stabilizers, and combination thereof.

i. Topical Excipients

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

a.) Lotions, Creams, Gels, Ointments, Emulsions, and Foams

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

4. Pulmonary Formulations

In one embodiment, the noscapine analogs are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorbtion occurs. The alveoli are covered by a thin epithelium without cilia or a mucus blanket and secrete surfactant phospholipids.

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solutions is water, physiologically acceptable aqueous solutions containing salts and/or buffers, such as phosphate buffered saline (PBS), or any other aqueous solution acceptable for administration to a animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the noscapine analogs. An appropriate solvent should be used that dissolves the noscapine analogs or forms a suspension of the noscapine analogs. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the noscapine analogs in the lungs and that the excipients that are present are present in amount that do not adversely affect uptake of noscapine analogs in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter, and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

### IV. Methods of Treatment

The anti-viral agents identified by the virtual screening methods or otherwise disclosed herein may be used in to reduce virus growth, infectivity, burden, shed, development of anti-viral resistance, and/or to enhance the efficacy of traditional anti-viral therapies.

In preferred embodiments, an effective amount of a compound identified in the virtual screening methods is used as an anti-viral agent.

### A. Nucleoprotein Binding

In some embodiments, compounds that bind the nucleoprotein are used as anti-viral agents. In a preferred embodiment, compound that bind influenza nucleoprotein are used as anti-influenza agents. In a more preferred embodiment, compounds that bind in the nucleozin binding site of influenza A are used as anti-viral agents to treat or prevent influenza A infection.

All viruses with negative-sense RNA genomes encode a single-strand RNA-binding nucleoprotein (NP). Nucleoproteins are proteins that are structurally associated with nucleic acid (either DNA or RNA). Influenza nucleoprotein is the most abundantly expressed protein during the course of infection with multiple functions including shuttling between the nucleus and the cytoplasm and encapsidation of the virus genome for RNA transcription, replication and packaging. NP interacts with a wide variety of both viral and host cellular macromolecules, including itself, RNA, the viral RNA-dependent RNA polymerase, and the viral matrix protein. NP also interacts with host polypeptides (such as actin), components of the nuclear import and export apparatus, and a nuclear RNA helicase.

The three potential binding novel binding sites on the influenza A NP include the small groove, the RNA binding pocket groove, and the tail loop groove.

In a preferred embodiment, anti-viral agents bind to the small groove (called the nucleozin binding groove) in the back of the body of influenza A nucleoprotein and involves residues 280 to 311 (VYGSAVASGYDFEREGYSLVGIDPFRLLQNSQ) (SEQ ID NO: 1). The secondary structure of these residues include three short helices (280˜287, 291˜294, and 301˜309) which are connected by loops formed by residues between helices. In this embodiment, the NP inhibitor is located in a small groove on the back of the body and interacts with residue N309 by hydrogen bond and Y289 by hydrophobic interaction, where the phenyl ring of compound is in parallel with the phenyl ring of Y289, and the distance between these two rings is between 3.2˜4.3 Å. In a preferred embodiment the NP inhibitor binds in the small groove, and the compound forms hydrogen bonds with residue S287. In some embodiments, the anti-viral agents can make binding contacts, alone or in combination with the above-listed contacts. In particular, anti-viral compounds can make contact with residues 465˜470 (sequence: ELSDEK) (SEQ ID NO: 2), residues 22˜26 (sequence: ATEIR) (SEQ ID NO: 3), residues A22˜47L (sequence: ATEIRASVGKMIDGIGRFYIQMCTEL) (SEQ ID NO: 4), R55, or combinations thereof.

In another embodiment, NP inhibitors bind to the RNA binding groove of the influenza A nucleoprotein. In this embodiment, the NP inhibitor is located in the RNA binding domain, which spans the interior groove between body and head of the nucleoprotein, and forms hydrogen bonds with residues Q364 and V363 that prohibit RNA from entering the arginine rich groove. Y148 was considered to be function as fixation of the first base of RNA.

In another embodiment, exemplary NP inhibitors bind to the tail loop groove of the influenza. In this embodiment, NP inhibitors are located in tail loop binding domain near to residue E339, and form hydrogen bonds with residues V186, R267, and G268. NP inhibitors in this binding pocket break the salt bridge formed between E339 and R416 from another monomer.

### B. Disorders to be Treated

Viral infections caused by both enveloped and non-enveloped viruses, including those that infect animals, vertebrates, mammals, and human patients can be prevented or treated with the compositions and methods described herein.

The compounds and methods are suitable for treating all viruses that infect vertebrates, particularly humans, and particularly viruses that are pathogenic in animals and humans. The viral infections and associated resultant diseases that can be treated include, but are not limited to CMV, RSV, arenavirus and HIV infections, and the diseases hepatitis, influenza, pneumonia, Lassa fever and AIDS. The International Committee on Taxonomy of Viruses contains a complete listing of viral strains, and is incorporated herein.

In some embodiments, the diseases to prevent or treat include viral infections. In preferred embodiments, the compounds and formulations are used to treat or prevent influenza A viral infections. Influenza A viruses that can be prevented or treated with formulations of the present method include H1N1, H2N2, H3N2, H5N1, H7N7, H1N2, H9N2, H7N2, H7N3, and H10N7. In preferred embodiments, the present formulations are useful for treatment of the influenza infection A strain caused by H1N1 or H3N2.

### C. Dosages

The dosage of an anti-viral formulation necessary to prevent viral growth and proliferation depends upon a number of factors including the types of virus that might be present, the environment into which the formulation is being introduced, and the time that the formulation is envisioned to remain in a given area.

Preferred compounds are those identified by a virtual screen. Exemplary compounds belong to formulae I-VI. Typical doses for treatment of viral infections are from about 0.1 mg to 250 mg per day per kilogram of subject by body weight.

The compounds can be administered to humans for the treatment of viral infection by either the oral or parenteral routes and may be administered orally at dosage levels of about 0.1 to about 500 mg/kg, preferably about 0.5 to 250 mg/kg/day given once or twice a day.

The present invention will be further understood by reference to following non-limiting examples.

### EXAMPLES

### Example 1

### Screening for Anti-Viral Agents

### Virus and Chemical Reagents

Influenza A/WSN/33, H3N2, and swine-origin influenza A (H1N1) virus S-OIV (A/HK/415742/09) were propagated in MDCK cells. After full cytopathic effects developed in cultures, in infected MDCK cell cultures, the viral particles were harvested and stored in −70° C. freezers for further studies. The influenza A virus strain A/Vietnam/1194/04 was grown in embryonated eggs and the virus-containing allantoic fluid was harvested and stored in aliquots at −70° C. A total of 50,240 structurally diverse small molecule compounds (ChemBridge Corporation, San Diego, Calif., USA) were screened. MTT (3-[4,5-Dimethylthiazol-2-yl]-2,5-diphenyltetrazolium bromide) was purchased from Sigma-Aldrich (USA). RNA oligomer (5′-UUUGUUACACACACACACGCUGUG-3′) used for RNA binding assays was synthesized by IDT (Integrated DNA Technologies).

### Cell-Based High Throughput Screening (HTS) in 384-Well Microtitre Plates

The primary HTS was carried out in a fully automated Beckman Coulter Core System (Fullerton, Calif., USA) integrated with a Kendro robotics CO₂ incubator (Thermo Fisher Scientific, Waltham, Mass., USA) at Chemical Genetics Unit, Department of Microbiology, Research Center of Infection and Immunology, LKS Faculty of Medicine, the University of Hong Kong. Compounds were arrayed in 384-well microtitre plates (Greiner Bio-One, Frickenhausen, Germany) in triplicate with a final concentration of 20 μg/ml and 5,000 MDCK cells per well in 50 μl complete Eagle's minimal essential medium (EMEM) supplemented with 1% heat-inactivated fetal bovine serum (FBS). Cells were then infected with influenza virus (A/WSN/33) at a multiplicity of infection (MOI) of 0.01. After infection, plates were incubated at 37° C. with 5% CO₂. At 3 days post-infection, 20 μl of 0.625 mg/ml of MTT was added into each well followed by an additional incubation time of 8 hour at 37° C. with 5% CO₂. At the end of the incubation, 30 μl of lauryl sulfate (SDS) with 0.01 M of hydrochloric acid (HCl) was added to solubilize the formazan, and after overnight incubation, MTT readings were recorded in a DTX 880 multimode detector (Beckman Coulter, USA) at 570 nm with 640 nm as the reference wavelength.

### Secondary Screening

Secondary screening was carried out in triplicate in 96-well tissue culture plate (TPP, Switzerland) at 10 μg/ml. Selected compounds were first dispensed in the wells, followed by the addition of 20,000 MDCK cells and 200 plaque forming units (PFU) of influenza A/WSN/33 (H1N1) virus into each well. The plates were incubated at 37° C. with 5% CO₂ and monitored daily using a Leica DM inverted light microscope (Wetzlar, Germany) for virus-induced cytopathic effect (CPE). Compounds that gave full protection of MDCK cells (no CPE) were selected for further studies. The cytotoxicity of selected compounds was determined by MTT assay according to manufacturer's instructions.

### Plaque Reduction Assay

The PRA assay was performed in triplicate in 24-well tissue culture plates (TPP, Switzerland). The MDCK cells were seeded at 1×10⁵ cells/well in EMEM (Invitrogen, Carlsbad, USA) with 10% FBS on the day before carrying out the assay. After 16 to 24 hours, 40-50 PFU of influenza virus were added to the cell monolayer with or without the addition of compounds and the plates further incubated for 2 hours at 37° C. with 5% CO₂ before removal of unbound viral particles by aspiration. The cell monolayer was washed once with EMEM before being overlaid with 1% low melting agarose (LMA) (Cambrex bioscience, Rockland, USA) in EMEM containing 1% FBS, 1 μg/ml TPCK trypsin (Invitrogen, Carlsbad, USA) and appropriate amounts of compound. The plates were incubated at 37° C. with 5% CO₂ for 72 hours. At 72 hours post-infection, the wells were fixed with 10% formaldehyde (BDH, Poole, England). After removal of the agarose plugs, the monolayers were stained with 0.7% crystal violet (BDH, Poole, England) and the plaques counted. The percentage of plaque inhibition relative to the control (without the addition of compound) plates were determined for each compound concentration, and the median effective concentration, EC₅₀, representing the concentration of a drug that is required for 50% inhibition in vitro, were calculated using Sigma plot (SPSS, USA). The PRA were carried out in triplicate and repeated twice for confirmation. For multicycle growth experiments for the evaluation of antiviral activities of compounds, 0.001 MOI was used accordingly and viral yield determined by plaque assay.

### Immunofluorescence Microscopy

A549 and MDCK Cells were grown to 70-80% confluency on coverslips. Cells were infected for 2 hours at MOI=10 and 5 for A549 and MDCK cells respectively in the presence or absence of 1 μM nucleozin and washed. Nucleozin was maintained in culture throughout the experiment. Infections were stopped at indicated time points by fixation in 4% paraformaldehyde (Electron Microscopy Sciences, Pa., USA) for 15 minutes. Cells were permeabilized in 0.1% Triton-X100 for 5 minutes and then were incubated for 1 hour with primary antibodies against NP (Abcam, Cambridge, UK) in PBS containing 5% goat serum (dilution 1:1000), washed and stained with FITC-conjugated secondary antibodies (Invitrogen, Ca, USA) (dilution 1:150) for 0.5 hour. Coverslips were then washed and counterstained with 4′,6-diamidino-2-phenylindole, dihydrochloride (DAPI)(Invitrogen, Ca, USA) for nucleus localization and mounted on slides using Prolong Gold antifade mounting medium (Invitrogen, Ca, USA) prior to image analysis by fluorescence microscopy (SPOT Diagnostic Instrument, MI, USA).

### Example 2

### Molecular Modeling of the Nucleozin Binding Site

In molecular docking study, all of nucleozin and NP complexes were obtained by Autodock 3.0.5. The files for docking were prepared by Autodock Tools. The docking calculations were carried out with the default genetic algorithm and Lamarckian genetic algorithm parameters, except for the following parameters, which were set to 150 individuals in population, 2,500,000 times of energy evaluation, 270,000 generations and 30 runs of docking. The docking grid box (X: 33.75 Å Y: 15.0 Å Z:15.0 Å) was centered in the nucleozin-binding groove and covered the whole nucleozin groove. Protein structures were downloaded from Protein Data Bank with homology modeling construction for unsolved structures. Currently the structures for some residues in influenza A viral NPs are not resolved yet, therefore the missing structures in nucleoprotein were constructed by Swiss-Model homology modeling sever in this investigation. In homology modeling, 2IQH and 2Q06 were taken as templates for H1N1 and H5N1 NP, respectively; and both 2WFS and 2IQH were templates for H3N2 NP structure, because the resolution of H3N2 NP structure is quite low and sequence of two structures are identical.

Results from molecular docking show the largest part of newly discovered nucleozin-binding groove involves residues 280˜311 (amino acid sequence: VYGSAVASGYDFEREGYSLVGIDPFRLLQNSQ), which are helices and loops. The secondary structure of these residues include three short helices 280˜287, 291˜294, and 301˜309, which are connected by loops formed by residues between helices. The location of residue Y289 is in the middle of these. Some proximal residues can also contribute interaction with ligands and proteins in the groove. These residues include loop residues 465˜470 (sequence: ELSDEK), a small part (residues 22˜26 sequence: ATEIR) of a long helix (residues A22˜47) (sequence: ATEIRASVGKMIDGIGRFYIQMCTEL) and R55. The residue R55 is pointing to the groove, which therefore makes it possible to bind with a ligand inside the groove. From the electrostatic surface of groove, more space exists on the side of loop residues 295˜300 than the other side of the groove.

Using the defined binding site on nucleoprotein, virtual screening indicates that nucleozin is a favorable ligand interacting influenza A nucleoproteins. Nucleozin interacts with H1N1 nucleoprotein residue N309 by hydrogen bond and Y289 by hydrophobic interaction, where the phenyl ring of compound is paralleling with phenyl ring of Y289 and the distances between these two rings are between 3.2˜4.3 Å. Virtual screening using H5N1 nucleoprotein indicates that nucleozin the benzene ring of nucleozin interacts with ring on Y289 by hydrophobic interaction and the nitro group on nucleozin can form hydrogen bonds with R55 and the distances of hydrogen bonds are 2.12 Å and 2.54 Å, respectively. Virtual screening using H53N2 nucleoprotein localize nucleozin molecule between residue N309 and Y289 in the binding site and the N and O on the five-member ring of nucleozin are hydrogen bonding with residue S287 and the distances of hydrogen bonds are 2.41 Å and 2.71 Å, respectively.

### Example 3

### In Vitro Evaluation of Nucleozin Binding Site Inhibitors

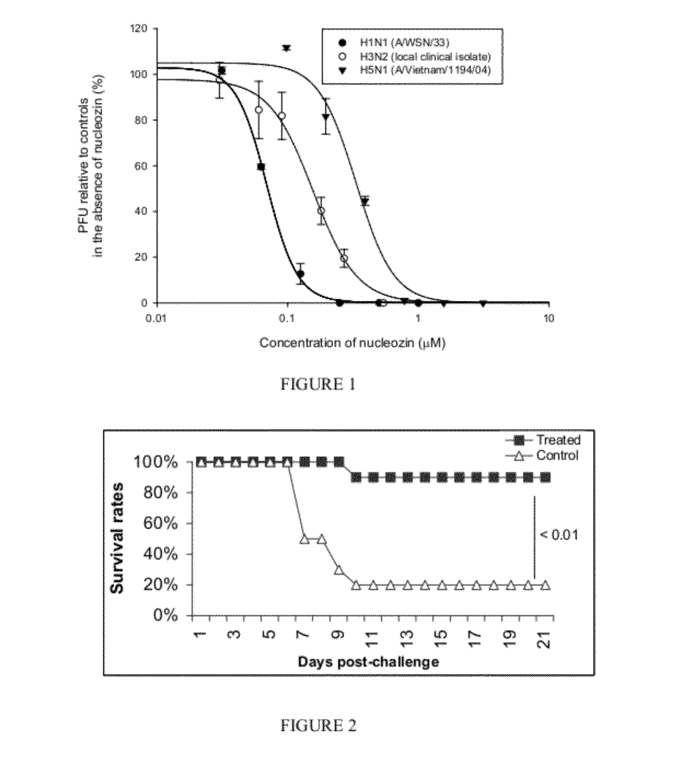

FIG. 1 shows a dose-response curve for nucleozin-treated mammalian cells infected with influenza A H1N1, H3N2, and H5N1 strains, graphing the percent plaque forming units (“PFU”) relative to controls in the absence of nucleozin as a function of the concentration of nucleozin (μM) for H1N1 (A/WSN/33) (filled circles), H3N2 (local clinical isolated) (open circles), and H5N1 (A/Vietnam/1194/04) (filled upside triangles).

### Example 4

### In Vivo Evaluation of Nucleozin Binding Site Inhibitors

Five to seven week old BALB/c female mice in biosafety level 3 housing were used that had access to standard pellet feed and water ad libitum. All experimental protocols followed the standard operating procedures of the approved biosafety level 3 animal facilities and were approved by the Animal Ethics Committee. One group (13 mice/group) of the mice was intraperitoneally (i.p.) injected with 100 μl of 2.5 mM of nucleozin (treated group) and the other group (13 mice) was injected with PBS (control group) one hour before inoculating the mice intranasally (i.n.) with 2×10⁴ TCID₅₀ of the A/Vietnam/1194/04 H5N1 virus in 20 μl 0.25 mM of the drug or PBS. We then gave 2 doses per day i.p. of 100 μl of 2.5 mM nucleozin or PBS for five days. Animal survival and general conditions were monitored for 21 days or till death. Statistical analysis of survival rate and viral load was performed by chi square test and the paired two-tailed Student's t test using Stata statistical software, respectively. Results were considered significant at P≦0.05. The results are shown in FIG. 2.

Mice treated with nucleozin had a significantly higher survival rate after inoculation by influenza A virus H5N1 strain A/Vietnam/1194/04 than untreated controls. Without any treatment, 80% died after 10 days post inoculation. In the treated group, 90% of animals receiving two doses of nucleozin (250 nmole per dose) per day for 5 days survived for more than 21 days.

Modifications and variations will be obvious to those skilled in the art from the foregoing detailed description and are intended to come within the scope of the appended claims.

We claim:

1. A virtual screening method of identifying anti-viral compounds that bind to a nucleoprotein binding site comprising:
a) obtaining the structural coordinates of a nucleoprotein;
b) applying 3-dimensional molecular modeling to the structural coordinates of the nucleoprotein binding pocket; and
c) screening spatial coordinates of the compound against the spatial coordinates of the nucleoprotein binding pocket to determine if the compound binds within the nucleoprotein binding pocket.

2. A virtual screening method for identifying anti-viral compounds that bind to influenza A NP nucleozin binding site comprising:
a) obtaining the structural coordinates of an influenza A NP;
b) applying three-dimensional molecular modeling to the structural coordinates of an influenza A NP binding pocket defined by the structural coordinates of at least amino acid residues 280 to 311; and
c.) screening spatial coordinates of the compound against the spatial coordinates of the influenza A NP binding pocket to determine if the compound binds within the influenza A NP binding pocket.

3. The method of claim 1, wherein the nucleozin binding site structure coordinates are X: 33.75 Å, Y: 15.0 Å, Z: 15.0 Å.

4. The method of claim 2, wherein the nucleozin binding site structure coordinates are X: 33.75 Å, Y: 15.0 Å, Z:15.0 Å.

5. The method of claim 1, wherein the compound forms a low energy, stable complex with the nucleoprotein.

6. The method of claim 2, wherein the compound forms a low energy, stable complex with the nucleoprotein.

7. A compound identified by the method of claim 1.

8. A compound identified by the method of claim 2.

9. An antiviral compound having one of the following chemical formulae:
Ar¹—Y—Ar²—X-Cy-Z—Ar³ (formula I)
wherein Ar¹, Ar², and Ar³ are each independently substituted or unsubstituted aryl or heteroaryl groups;
X, Y, and Z are independently absent (i.e, a direct bond) or selected from —C(═O)—, —S(═O)—, —SO₂—, —C(═O)N(R₁), —N(R₂)—, —C(R₃)═C(R₄)—, and —C(R₅R₆)ₙ—;
n is 0 to 10;
R₁-R₆ are each independently selected from hydrogen; halogen; hydroxy;
nitro; nitrile; isonitrile; urea; guanidine; cyano; formyl, acyl, carboxyl; thioester, thioacetate, thioformate; primary, secondary, or tertiary amine; amide; amidine; imine; azide; thiol, substituted or unsubstituted thioalkyl; isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl; and
Cy is a 5-7 membered substituted or unsubstituted cyclic or heterocyclic group; 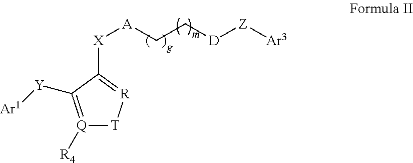
wherein Ar¹ and Ar³ are each independently substituted or unsubstituted aryl or heteroaryl groups;
X, Y, and Z are independently absent or selected from the group consisting of —C(═O)—, —S(═O)—, —SO₂—, —C(═O)N(R₁₀), —C(R₁₂)═C(R₁₃)—, and —C(R₁₄R₁₅)ₙ—,
n, g, and m are independently 0 to 10;
T, Q, and R are, as valence and stability permit, independently selected from C(R₈R₉), nitrogen, oxygen, phosphorous, silicon, and arsenic;
A and D are each independently CR₁₆R₁₇ or NR₁₈;
wherein R₄ and R₈-R₁₈ independently are absent, or are selected from hydrogen; halogen; hydroxy; nitro; nitrile; isonitrile; urea; guanidine; cyano; formyl, acyl, carboxyl; thioester, thioacetate, thioformate; primary, secondary, or tertiary amine; amide; amidine; imine; azide; thiol, substituted or unsubstituted thioalkyl; isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl or wherein —CR₁₅R₁₆—, —NR₁₇—, or combinations thereof, when taken together with the optional bridging methylene groups, form a 5-8-membered cyclic structure; 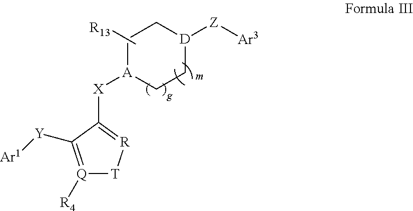
wherein Ar¹ and Ar³ are each independently substituted or unsubstituted aryl or heteroaryl groups;
X, Y, and Z are independently absent or selected from the group consisting of —C(═O)—, —S(═O)—, —C(═O)N(R₁₀), —N(R₁₁)—, —C(R₁₂)═C(R₁₃)—, and —C(R₁₄R₁₅)ₙ—,
n, g, and m are independently 0 to 10;
A, D, T, Q, and R are, as valence and stability permit, independently selected from C(R₈R₉), nitrogen, oxygen, phosphorous, silicon, and arsenic;
wherein R₄ and R₈-R₁₅ independently are absent, or are selected from hydrogen; halogen; hydroxy; nitro; nitrile; isonitrile; urea; guanidine; cyano; formyl, acyl, carboxyl; thioester, thioacetate, thioformate; primary, secondary, or tertiary amine; amide; amidine; imine; azide; thiol, substituted or unsubstituted thioalkyl; isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl; 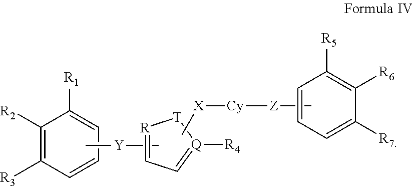
wherein X, Y, and Z are independently absent or selected from the group consisting of —C(═O)—, —S(═O)—, —SO₂—, —C(═O)N(R₁₀), —N(R₁₁)—, —C(R₁₂)═C(R₁₃)—, and —C(R₁₄R₁₅)ₙ—;
wherein n is 0 to 10;
T, Q, and R are, as valence and stability permit, independently selected from C(R₈R₉), nitrogen, oxygen, phosphorous, silicon, and arsenic; and
Cy is a 4-7 membered substituted or unsubstituted cyclic or heterocyclic group;
wherein R₁-R₁₅ independently are absent, or are selected from hydrogen; halogen; hydroxy; nitro; nitrile; isonitrile; urea; guanidine; cyano; formyl, acyl, carboxyl; thioester, thioacetate, thioformate; primary, secondary, or tertiary amine; amide; amidine; imine; azide; thiol, substituted or unsubstituted thioalkyl; isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl; 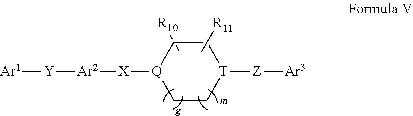
wherein Ar¹, Ar², and Ar³ are each independently substituted or unsubstituted aryl or heteroaryl groups;
X, Y, and Z are independently absent or selected from the group consisting of —C(═O)—, —S(═O)—, —SO₂—, —C(═O)N(R₁), —N(R₂)—, —C(R₃)═C(R₄)—, and —C(R₅R₆)ₙ—;
n, g, and m are independently 0 to 10;
Q and T are independently selected from nitrogen or CR₇; and
R₁-R₇, R₁₀, and R₁₁ are independently selected from hydrogen; halogen; hydroxy; nitro; nitrile; isonitrile; urea; guanidine; cyano; formyl, acyl, carboxyl; thioester, thioacetate, thioformate; primary, secondary, or tertiary amine; amide; amidine; imine; azide; thiol, substituted or unsubstituted thioalkyl; isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl; or 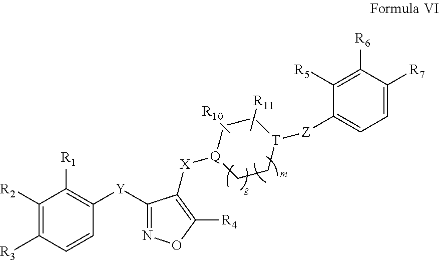
wherein X, Y, and Z are independently absent or selected from the group consisting of —C(═O)—, —S(═O)—, —SO₂—, —C(═O)N(R₁₂), —N(R₁₃)—, —C(R₁₄)═C(R₁₅)—, and —C(R₁₆R₁₇)ₙ—,
n, g, and m are independently 0 to 10;
Q and T are independently selected from nitrogen or CR₁₈; and
R₁-R₁₈ are independently selected from hydrogen; halogen; hydroxy; nitro; nitrile; isonitrile; urea; guanidine; cyano; formyl, acyl, carboxyl; thioester, thioacetate, thioformate; primary, secondary, or tertiary amine; amide; amidine; imine; azide; thiol, substituted or unsubstituted thioalkyl; isocyanate; isothiocyanate; phosphoryl; phosphate; phosphinate; sulfate; sulfonate; sulfamoyl; sulfonamide; sulfonyl; substituted or unsubstituted linear or branched alkyl, alkenyl, or alkynyl; substituted or unsubstituted linear or branched alkoxy; substituted or unsubstituted C₃-C₁₀ cycloalkyl, cycloalkenyl, heterocyloalkyl, or heterocycloalkenyl; substituted or unsubstituted aryl or heteroaryl.

10. The compound of Formula I in claim 9, wherein Ar¹ is substituted with hydrogen, hydroxyl, nitro, amino, or azide; Ar² is substituted with a methyl group; X is C═O; Y and Z are absent; and Ar³ is substituted with a halo group, a nitro group, or a combination of a halo and nitro group.

11. The compound of claim 10, wherein Cy is a substituted 5-7 membered unsaturated ring containing 2 nitrogen atoms, wherein one nitrogen atom is bonded to X and another nitrogen atom is bonded to Z.

12. The compound of claim 11, wherein Cy is a substituted piperazine, wherein one nitrogen is bonded to X and the second nitrogen is bonded to Z.

13. The compound of Formula II in claim 9, wherein Ar¹ is substituted with hydrogen, hydroxyl, nitro, amino, or azide; X is C═O; Y and Z are absent, and Ar³ is substituted with a halo group, a nitro group, or a combination of a halo and nitro group.

14. The compound of claim 13, wherein R₄ is methyl.

15. The compound of claim 14, wherein Q is carbon, T is oxygen, and R is nitrogen.

16. The compound of claim 15, wherein g and m are 1.

17. The compound of claim 16, wherein and A and D are NR₁₅, and wherein R₁₅—R₁₅ represents a —CH₂—CH₂— linker, such that A-D defines a piperazine.

18. The compound of Formula III in claim 9, wherein Ar¹ is substituted with hydrogen, hydroxyl, nitro, amino, or azide; X is C═O; Y and Z are absent, and Ar³ is substituted with a halo group, a nitro group, or a combination of a halo and nitro group.

19. The compound of claim 18, wherein Q is carbon, T is oxygen, and R is nitrogen.

20. The compound of claim 19, wherein A and D are nitrogen.

21. The compound of claim 20, wherein R₄ and R₁₃ are independently hydrogen or methyl.

22. The compound of claim 21, wherein R₄ is methyl and R₁₃ is hydrogen.

23. The compound of Formula IV in claim 9, wherein Cy is a substituted 5-7 membered unsaturated ring containing 2 nitrogen atoms, wherein one nitrogen atom is bonded to X and another nitrogen atom is bonded to Z.

24. The compound of claim 23, wherein Cy is a substituted piperazine, wherein one nitrogen is bonded to X and the second nitrogen is bonded to Z, Y and Z are absent, X is C═O, T is oxygen, Q is carbon, and R is nitrogen.

25. The compound of claim 24, wherein R₁-R₃ and R₅-R₇ are selected from a halo group, a nitro group, or a combination of a halo and nitro group.

26. The compound of claim 25, wherein R₄ is a methyl group.

27. The compound of Formula V in claim 9, wherein Q and T are both nitrogen.

28. The compound of claim 27, wherein R₁₀ is a methyl group and R₁₁ is hydrogen.

29. The compound of claim 28, wherein R₁₀ and R₁₁ are both hydrogen.

30. The compound of claim 29, wherein Y and Z are absent, g and m are 1, and X is C═O.

31. The compound of claim 30, wherein Ar¹ and Ar³ are a substituted phenyl, Ar² is a substituted isoxazole.

32. The compound of Formula VI in claim 9, wherein Q and T are both nitrogen.

33. The compound of claim 32, wherein Y and Z are absent and X is C═O.

34. The compound of claim 33, wherein R₁₀ is a methyl group and R₁₁ is hydrogen.

35. The compound of claim 34, wherein g and m are 1.

36. The compound of claim 35, wherein R₁-R₃ and R₅-R₇ are selected from a halo group, a nitro group, or a combination of a halo and nitro group.

37. A compound with the following structure:
[4-(2-chloro-4-nitro-phenyl)-piperazin-1-yl]-[3-(4-hydroxy-phenyl)-5-methylisoxazol-4-yl]-methanone;
[4-(2-chloro-4-nitro-phenyl)-piperazin-1-yl]-[3-phenyl-5-methyl-isoxazol-4-yl]-methanone;
[4-(2-chloro-4-nitro-phenyl)-piperazin-1-yl]-[3-(4-amino-phenyl)-methylisoxazol-4-yl]-methanone;
[4-(2-chloro-4-nitro-phenyl)-piperazin-1-yl]-[3-(4-azido-phenyl)-5-methylisoxazol-4-yl]-methanone;
[4-(2-chloro-4-nitro-phenyl)-piperazin-1-yl]-[3-(2-chloro-phenyl)-5-methylisoxazol-4-yl]-methanone;
[4-(2-chloro-4-nitro-phenyl)-2-methyl-piperain-1-yl]-[3-(2-chloro-phenyl)-5-methyl-isoxazol-4-yl]-methanone;
[4-(2-chloro-4-nitro-phenyl)-2-methyl-piperain-1-yl]-[3-phenyl-5-methylisoxazol-4-yl]-methanone;
[4-(4-nitro-phenyl)-piperazin-1-yl]-[3-(2-chloro-phenyl)-5-methyl-isoxazol-4-yl]-methanone;
and [4-(4-nitro-phenyl)-piperazin-1-yl]-[3-(2,6-dichloro-phenyl)-5-methyl-isoxazol-4-yl]-methanone.

38. A formulation comprising the compound of claim 9 in an amount effective for treating or preventing a viral infection.

39. A method for treating or preventing viral infection in a patient in need thereof comprising administering to the patient a formulation comprising an effective amount of the compound of claim 9.

40. The method of claim 39, wherein the viral infection is influenza A selected from the group of strains consisting of H1N1, H3N2, and H5N1.

41. The method of claim 40, wherein the formulation provides a dosage from about 0.1 mg to about 250 mg per day per kilogram of body weight for an adult human.

42. The method of claim 41, wherein the dosage is about 250 mg per day.


SEQUENCE LISTING (12 sequences)

SEQ ID NO: 1 - protein, 32 aa, Influenza A virus
VYGSAVASGYDFEREGYSLVGIDPFRLLQNSQ

SEQ ID NO: 2 - protein, 6 aa, Influenza A virus
ELSDEK

SEQ ID NO: 3 - protein, 5 aa, Influenza A virus
ATEIR

SEQ ID NO: 4 - protein, 26 aa, Influenza A virus
ATEIRASVGKMIDGIGRFYIQMCTEL

SEQ ID NO: 5 - DNA, 1565 bp, Influenza A virus
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 6 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

SEQ ID NO: 7 - DNA, 1566 bp, Influenza A virus
agcaaaagcagggttaataatcactcaccgagtgacatcaaaatcatggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgatcatgaagggcggttgatccagaacagcttgacaatagagaaaatggtgctctctgcttttgatgaaagaaggaataaatacctggaagaacaccccagcgcggggaaagatcccaagaaaactggggggcccatatacaggagagtagatggaaaatggatgagggaactcgtcctttatgacaaagaagagataaggcgaatctggcgccaagccaacaatggtgaggatgcgacagctggtctaactcacataatgatctggcattccaatttgaatgatgcaacataccagaggacaagagctcttgttcgaactggaatggatcccagaatgtgctctctgatgcagggctcgactctccctagaaggtccggagctgcaggtgctgcagtcaaaggaatcgggacaatggtgatggaactgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattgatattgagagggtcagttgctcacaaatcttgcctacctgcctgtgcgtatggacctgcagtatccagtgggtacgacttcgaaaaagagggatattccttggtgggaatagaccctttcaaactacttcaaaatagccaaatatacagcctaatcagacctaacgagaatccagcacacaagagtcagctggtgtggatggcatgccattctgctgcatttgaagatttaagattgttaagcttcatcagagggacaaaagtatctccgcgggggaaactgtcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaaggacttcagacatgagggcagaaatcataagaatgatggaaggtgcaaaaccagaagaagtgtcattccgggggaggggagttttcgagctctcagacgagaaggcaacgaacccgatcgtgccctcttttgatatgagtaatgaaggatcttatttcttcggagacaatgcagaagagtacgacaattaaggaaaaaatacccttgtttctact

SEQ ID NO: 8 - protein, 498 aa, Influenza A virus
MASQGTKRSYEQMETDGDRQNATEIRASVGKMIDGIGRFYIQMCTELKLSDHEGRLIQNSLTIEKMVLSAFDERRNKYLEEHPSAGKDPKKTGGPIYRRVDGKWMRELVLYDKEEIRRIWRQANNGEDATAGLTHIMIWHSNLNDATYQRTRALVRTGMDPRMCSLMQGSTLPRRSGAAGAAVKGIGTMVMELIRMVKRGINDRNFWRGENGRKTRSAYERMCNILKGKFQTAAQRAMVDQVRESRNPGNAEIEDLIFLARSALILRGSVAHKSCLPACAYGPAVSSGYDFEKEGYSLVGIDPFKLLQNSQIYSLIRPNENPAHKSQLVWMACHSAAFEDLRLLSFIRGTKVSPRGKLSTRGVQIASNENMDNMGSSTLELRSGYWAIRTRSGGNTNQQRASAGQTSVQPTFSVQRNLPFEKSTIMAAFTGNTEGRTSDMRAEIIRMMEGAKPEEVSFRGRGVFELSDEKATNPIVPSFDMSNEGSYFFGDNAEEYDN

SEQ ID NO: 9 - DNA, 1565 bp, Influenza A virus
agcaaaagcagggtagataatcactcaccgagtgacatcaacatcatggcgtctcaaggcaccaaacgatcttatgaacagatggaaactggtggagaacgccagaatgctactgagattagggcatctgttggaagaatggttagtggcattgggaggttctacatacagatgtgcacagaactcaaactcagtgactatgaagggaggctgatccagaacagcataacaatagagagaatggtgctctctgcatttgatgaaagaaggaacagatacctggaagaacaccccagtgcggggaaggacccgaagaaaactggaggtccaatttatcggaggagagacgggaaatgggtgagagagctgattctgtatgacaaagaggagatcaggaggatttggcgtcaagcgaacaatggagaggacgcgactgctggtcttacccacctgatgatatggcattccaacctaaatgatgccacatatcagagaacgagagctctcgtgcgtactggaatggatcccaggatgtgctctctgatgcaaggatcaactctcccgaggagatctggagctgccggtgcagcagtaaagggggtagggacaatggtgatggagctgattcggatgataaagcgagggatcaacgaccggaatttctggagaggtgaaaatggaagaagaacaaggattgcatatgagagaatgtgtaacatcctcaaagggaaattccaaacagcagcacaaagagcaatgatggatcaagtgcgagagagcagaaatcctgggaatgctgaaattgaagatctcatttttctggcacggtctgcactcatcctgagaggatcagtggcccataaatcctgcttgcctgcttgtgtgtacggacttgcagtggccagtggatatgactttgagagagaagggtactctctggttggaatagatcctttccgtctgcttcaaaacagccaggtctttagtctcattagaccaaatgagaatccagcacataagagtcaattagtgtggatggcatgccactctgcagcatttgaggaccttagagtctcaagtttcatcagagggacaagagtggtcccaagaggacagctatccaccagaggggttcaaattgcctcaaatgagaacatggaagcaatggactccaacactcttgaactgagaagtagatattgggctataagaaccagaagcggaggaaacaccaaccagcggagggcatctgcaggacagatcagcgttcagcccactttctcagtacagagaaatcttcccttcgaaagagcaaccattatggcagcatttacagggaatactgagggcagaacgtctgacatgaggactgaaatcataggaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaaagaaaaatacccttgtttctact

SEQ ID NO: 10 - protein, 498 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQRRASAGQISVQPTFSVQRNLPFERATIMAAFTGNTEGRTSDMRTEIIGMMESARPEDVSFQGRGVFELSDEKATNPIVPSFDMNNEGSYFFGDNAEEYDN

SEQ ID NO: 11 - DNA, 1515 bp, Influenza A virus
tgagtgacatcaacatcatggcgtctcaaggcaccaaacgatcttatgaacagatggaaactggtggagaacgccagaatgccactgagatcagggcatccgttggaagaatggttggtggaattgggaggttttacatacagatgtgcactgaactcaaactcagcgaccaggaaggaaggttgatccagaacagtataacagtagagagaatggttctctctgcatttgatgaaaggaggaacaggtacctagaggaacatcccagtgcggggaaggacccgaagaagaccggaggtccaatctaccgaagaagaaacgggaaatgggtgagagagctgattctgtatgacaaagaggagataaggagaatttggcgccaagcgaacaatggagaagacgcaactgctggtctcactcacatgatgatttggcattccaacctaaatgatgccacataccagagaacaagagccctcgtgcggactggaatggaccccagaatgtgctctctgatgcaaggatcaaccctcccgaggagatctggagctgctggtgcagcaataaagggagtcgggacgatggtaatggaactaattcggatgataaagcgaggcattaatgaccggaacttctggagaggcgagaatggacgaagaacaaggattgcatatgagagaatgtgcaacatcctcaaagggaaatttcaaacagcagcacaaaaagcaatgatggatcaggtgcgagaaagcagaaatcctgggaatgctgaaattgaagatctcatttttctggcacggtctgcactcatcctgagaggatccgtagcccataagtcctgcttgcctgcttgtgtgtacggactcgctgtggccagtggatatgattttgagagggaagggtactctctggttgggatagatcctttccgtctgcttcagaacagtcaggtcttcagtctcattagaccaaaagagaatccagcacataaaagtcaattggtatggatggcatgccattctgcagcatttgaggacctgagagtctcaagtttcattagaggaacaagagtaatcccaagaggacaactatccactagaggagttcagattgcttcaaatgagaacgtggaagcaatggactccagcactcttgaactgagaagcagatattgggctataaggaccaggagtggaggaaacaccaaccaacagagagcatctgcaggacaaatcagtgtacagcccactttctcagtacagagaaatcttcccttcgaaagagtgaccattatggccgcgtttaaggggaataccgagggcagaacatctgacatgaggactgaaatcataagaatgatggaaagtgccagaccagaagatgtgtctttccaggggcggggagtcttcgagctctcagacgaaaaggcaacgaacccgatcgtgccttcctttgacatgagtaatgaaggatcttatttcttcggagacaatgcagaggaatatgacaattgaa

SEQ ID NO: 12 - protein, 498 aa, Influenza A virus
MASQGTKRSYEQMETGGERQNATEIRASVGRMVGGIGRFYIQMCTELKLSDQEGRLIQNSITVERMVLSAFDERRNRYLEEHPSAGKDPKKTGGPIYRRRNGKWVRELILYDKEEIRRIWRQANNGEDATAGLTHMMIWHSNLNDATYQRTRALVRTGMDPRMCSLMQGSTLPRRSGAAGAAIKGVGTMVMELIRMIKRGINDRNFWRGENGRRTRIAYERMCNILKGKFQTAAQKAMMDQVRESRNPGNAEIEDLIFLARSALILRGSVAHKSCLPACVYGLAVASGYDFEREGYSLVGIDPFRLLQNSQVFSLIRPKENPAHKSQLVWMACHSAAFEDLRVSSFIRGTRVIPRGQLSTRGVQIASNENVEAMDSSTLELRSRYWAIRTRSGGNTNQQRASAGQISVQPTFSVQRNLPFERVTIMAAFKGNTEGRTSDMRTEIIRMMESARPEDVSFQGRGVFELSDEKATNPIVPSFDMSNEGSYFFGDNAEEYDN